(19) United States
(12) Patent Application Publication
(10) Pub. No.: US 2015/0037399 A1
(43) Pub. Date: Feb. 5, 2015

(54) NOVEL USE OF PIPOXOLAN AND ITS PHARMACEUTICAL COMPOSITION

(71) Applicant: China Medical University, Taichung City (TW)

(72) Inventors: Yuh-Fung CHEN, Taichung City (TW); Huei-Yann TSAI, Taichung City (TW)

(73) Assignee: China Medical University, Taichung City (TW)

(21) Appl. No.: 14/267,932

(22) Filed: May 2, 2014

(30) Foreign Application Priority Data
Aug. 2, 2013 (TW) 102127800

(51) Int. Cl. A61K 31/4525

(52) U.S. Cl. CPC A61K 31/4525
USPC 424/450; 514/326

(57) ABSTRACT

The present disclosure concerns the novel use of pipoxolan and its pharmaceutical composition. Pipoxolan is useful therapeutic drugs for pathological conditions caused by vascular smooth muscle cell proliferation and migration to relieve a body appeared vascular injury, cerebrovascular ischemia, intimal hyperplasia, atherosclerotic stenosis, cerebral ischemia, and stroke.

### RELATED APPLICATIONS

This application claims priority to Taiwan Application Serial Number 102127800, filed Aug. 2, 2013, which is herein incorporated by reference.

### BACKGROUND

1. Technical Field

The present disclosure relates to the novel use of pipoxolan. More particularly, the present disclosure relates to the novel use of pipoxolan and pharmaceutical compositions thereof for treating a pathological condition caused by proliferation and migration of vascular smooth muscle cells.

2. Description of Related Art

Pipoxolan HCl (5,5-diphenyl-2-(2-piperidin-1-ylethyl)-1,3-dioxolan-4-one hydrochloride), a 1,3-dioxolane derivative also known as rowapraxin, was originally synthesized by Pailer et al. in 1968. It has anti-spasmodic effects.

Pipoxolan is used clinically to relieve smooth muscle spasms, and it is effective in treating dysmenorrhea, renal colic, bilateral urinary lithiasis, cholelithiasis, chronic gastritis, post-natal uterine pain, urolithiasis and hydronephrosis.

Cerebrovascular accident or atherosclerosis usually results in intimal thickening. Hence atherosclerotic stenosis or occlusion of a major cerebral artery is a leading cause of stroke.

In damaged vessels, vascular smooth muscle cells (VSMCs) migrate from the membrane into the intima and induce intimal hyperplasia. Abnormal VSMCs proliferation and migration cause thickening of endothelial cells result in restenosis after balloon angioplasty or coronary stenting. However, vascular injury can only be controlled by medication or surgery, but restenosis can occur months or years after injury and initial treatment to date.

### SUMMARY

According to one embodiment of the present disclosure, a method for treating pathological condition caused by proliferation and migration of VSMCs includes administering an effective amount of pipoxolan.

According to another embodiment of the present disclosure, a pharmaceutical composition for reducing cerebrovascular pathological condition in a subject includes a therapeutically effective amount of pipoxolan HCl and pharmaceutically acceptable carrier.

### BRIEF DESCRIPTION OF THE DRAWINGS

The present disclosure can be more fully understood by reading the following detailed description of the embodiment, with reference made to the accompanying drawings as follows:

FIG. 1 is photomicrograph showing the effects of pipoxolan on cerebral infarction of rats according to experiment I of the present disclosure;

FIG. 2 is bar chart illustrating different levels of the effects of pipoxolan on cerebral infarction of rats according to experiment I of the present disclosure;

FIG. 3 illustrates the effects of pipoxolan on carotid-ligation induced intimal hyperplasia in mice according to experiment II of the present disclosure;

FIG. 4 illustrates the effects of pipoxolan on PDGF-BB-induced VSMCs migration according to wound healing assay of the present disclosure;

FIG. 5 illustrates the effects of pipoxolan on PDGF-BB-induced VSMCs migration according to transwell assay of the present disclosure;

FIG. 6A represents the western blotting results of VSMCs migration related proteins according to experiment IV of the present disclosure;

FIG. 6B is bar chart illustrating protein expression levels of Ras, MEK, and p-MEK according to experiment IV of the present disclosure;

FIG. 6C is bar chart illustrating protein expression levels of ERK and p-ERK according to experiment IV of the present disclosure;

FIG. 6D is bar chart illustrating protein expression levels of MMP-2, and MMP-9 according to experiment IV of the present disclosure.

### DETAILED DESCRIPTION

A novel use of pipoxolan is provided for treating pathological condition caused by proliferation and migration of VSMCs. According to earlier results of regardless in vitro whether in vivo VSMCs migration experiment models, aforementioned pipoxolan is capable of reducing intimal hyperplasia caused by VSMCs migration and proliferation. Furthermore, pipoxolan is protective in models of ischemia-reperfusion-induced cerebral infarction and carotid artery ligation-induced intimal hyperplasia. Thus pipoxolan is a potentially effective prevention therapeutic agent for cerebrovascular disease. The following are descriptions of the specific terms used in the specification:

The terms “compounds”, “compositions”, “active compounds”, “agent” or “medicament” can be used alternatively. The term means that a compound or composition can induce pharmaceutical and/or physiological reactions through local and/or systemic effects when it administered to an object (human or animal).

The term “subject” or “patient” refers to an object who accepts the treatment compound and/or method according to the present disclosure. The term “subject” or “patient” covers male and female unless otherwise specified. Moreover, the term “subject” or “patient” includes any mammal, preferably human; it can be benefited by compound treatment as described.

The term “administered”, “administering” or “administration” refers to the direct administration of a compound or composition, or administration of prodrug, derivative or analog of the active compound, which can be formed an amount of the active compound in the body of a subject.

The term “treating” refers to administer a composition to cells or a subject for preventive (eg, prophylaxis), healing or palliative treatment. The subject may have intimal thickening which caused by VSMCs migration and proliferation result in the risk of pathological changes, medical disorder, symptom, disease, disorder associated with abnormality or susceptible to a disease. The term “treating” means partially or completely alleviates, ameliorates, reduces one or more symptoms or features of specific abnormality or disease, or delays disease occurrence, impedes disease progress and reduces disease severity and/or reduces disease incidence. The term “treating” also means treatment for a subject who may not appear any signs of disease or has signs of disease or disorder and/or condition in order to reduce the risk of developing pathological changes associated with the disease, disorder and/or condition. In the present specification, the treatment is considered “effective” when one or more symptoms or clinical indicators are reduced. Alternatively, an effective treatment means reduced or suspended the disease progress. The “treating” includes not only improving symptoms or reducing disease indicators, also includes stopping or slowing down highly disease progression or worsening symptoms which may occur when a subject without treatment. Beneficial clinical results include one or more symptoms alleviating, the stability of the disease state, reducing of disease extent, delaying or slowing the disease process, partially or completely amelioration or palliation of the disease state. The above description includes the detection or non-detection of symptoms, extent, state or process.

The term “an effective amount” refers to the dosage amount of a composition, and the dosage amount is suitable for administration to achieve the desired therapeutic effect or response after a period. The term “therapeutically effective amount” refers to the therapeutic agent sufficient of the pharmaceutical composition to produce a desired “therapeutically effective” which is defined above. The specific therapeutically effective amount depends on various factors such as the particular condition to be treated, the individual physiological conditions (e.g., body weight, age or sex), the type of mammal or animal being treated, the duration of the treatment, the current therapy (if any) and the structure of the particular formulation, the compound or its derivative. The term “an effective amount” also refers to under this dosage amount the toxicity or negative effect of the compound or composition is less than its positive effects.

A person who has ordinarily skilled in the art of the present disclosure can base on animal tests administered dose of this description to calculate the human equivalent dose (human equivalent dose, HEQ) by according to criteria such as the U.S. Food and Health Administration proposed (Estimating the Maximum Safe Starting Dose in Initial Clinical Trials for Therapeutics in Adult Healthy Volunteers).

Reference will now be made in detail to the present embodiments of the present disclosure, examples of which are illustrated in the accompanying drawings. Wherever possible, the same reference numbers are used in the drawings and the description to refer to the same or like parts.

### Examples

I. Pipoxolan Effects of Pipoxolan on Cerebral Infarction Area in the Brain of Ischemic Rats

(1) Animals

Animals are male Sprague-Dawley (SD) rats, weighing 225-275 g and male ICR mice, weighing 25-30 g were purchased from BioLASCO Co. Ltd. (Taipei, Taiwan). All animals were fed regular chow and housed in standard cages at a constant temperature of 22±1° C. and relative humidity of 55±5% with 12 hr inverted light-dark cycle for 1 week prior to the experiments. Surgery was performed under zoletil® anesthesia. Five to six animals were used to obtain consistent data in each group. Pipoxolan was dissolved in dimethylsulfoxide (DMSO) and diluted in tissue culture medium before use.

(2) Transient Focal Cerebral Ischemia—Reperfusion Model

Focal ischemia was induced by occlusion of both common carotid arteries (CCA) and the right middle cerebral artery (MCA). Eighteen rats were randomly assigned to 3 treatment groups, six rats for each group. Group 1 rats were treated with saline and groups 2 and 3 treated with pipoxolan (10 mg/kg and 30 mg/kg, respectively). Rats were fasted overnight with free access to water. Pipoxolan (10, 30 mg/kg) was orally administered 1 hr prior to transient focal cerebral ischemia-reperfusion. Animals then were anesthetized with zoletil® (25 mg/kg i.p.), and the skull was exposed, and a small burr hole was produced over the MCA. Beneath the right MCA, proximal to the major bifurcation of the right MCA, distal to the lenticulostriate arteries, and rostral to the rhinal fissure, a 10-0 nylon monofilament was placed. The artery then was lifted and the nylon filament rotated clockwise. Both CCA were then occluded using a microvascular clip. After 90 minutes of occlusion, reperfusion was established by removing the microvascular clips from the CCA, rotating the nylon monofilament counterclockwise and removing it from beneath the MCA. After 24 hr, all rats were alive and neurobehavioral deficits evaluation and infarct assessment were performed

(3) Evaluation of Neurological Deficits and Assessment of Infarction

Twenty four hours following reperfusion, neurological function of rats was assessed. The degree of neurological deficits were divided 0-5 (higher score is for more severe neurological deficits) as follows: 0=no neurological deficits, 1=failure to extend left forepaw fully, 2=circling to the left, 3=falling to left, 4=no spontaneous walking with a depressed level of consciousness, and 5=death. Neurological evaluation was done by an observer who was blind to the treatment conditions of the animals. After completion of the neurobehavioral evaluation, rats were deeply anesthetized by an intraperitoneal injection of zoletil® (50 mg/kg) followed by intracardiac perfusion with 200 ml of freezing PBS and animals were then decapitated. The brain was removed and sliced in 2-mm sections using a rodent brain matrix slicer. The sections were stained with 2% 2,3,5-triphenyltetrazoliumchloride (TTC) for 10 min at 37° C. and fixed in 10% formalin. The image of each section was digitized and the infarct area was determined morphometrically using Image-Pro Plus 6.0 (Media Cybernetics, Md., USA).

(4) TUNEL Assay

Nine rats were randomly assigned into 3 treatment groups, three rats for each group. Group 1 rats were treated with saline and groups 2 and 3 treated with pipoxolan (10 mg/kg and 30 mg/kg, respectively). Focal ischemia was induced by occlusion of both CCA and the right MCA as previously described. Twenty four hours after focal ischemia, rats were deeply anesthetized by intraperitoneal injection of 50 mg/kg of zoletil® followed by intracardiac perfusion with 200 ml of 0.9% saline, followed by 4% paraformaldehyde in 0.1 M PBS and animals were decapitated. The percentage of positive TUNEL staining cells within areas of the cortex was estimated.

(5) Immunohistochemical Staining of Cleaved Capase-3

Rat brain slices were incubated with anti-caspase-3 antibodies (GTX73093, GeneTex Inc., USA) overnight and immunohistochemical labeled using a NovoLink Polymer Detection System Kit (Leica Microsystems Inc., Newcastle Upon Tyne, UK). The percentage of positive caspase-3 staining cells within the cortex was estimated based on the average number of cells in a defined area.

Results

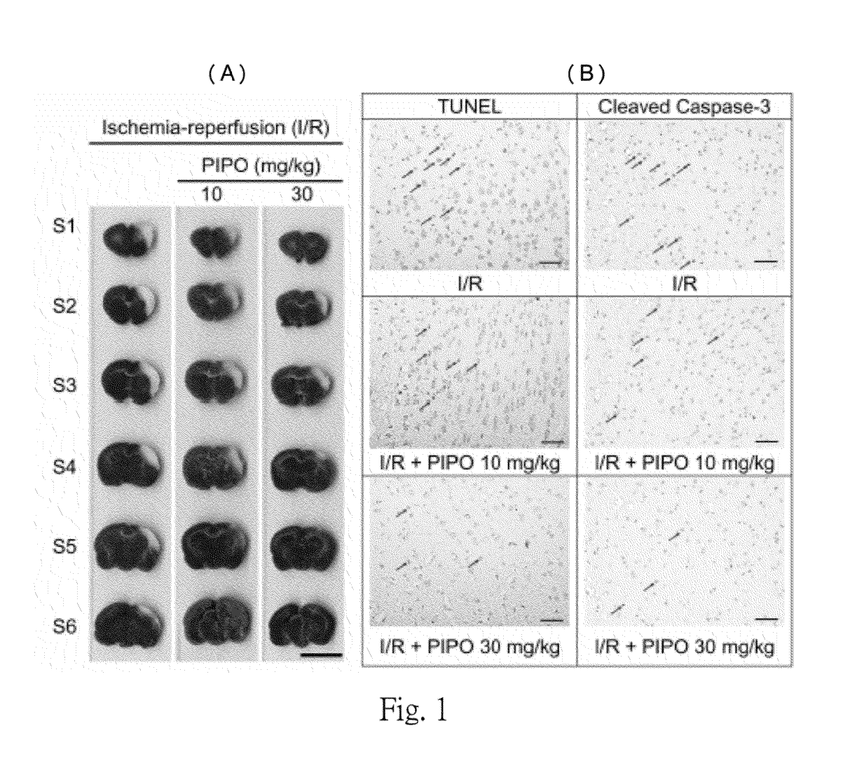

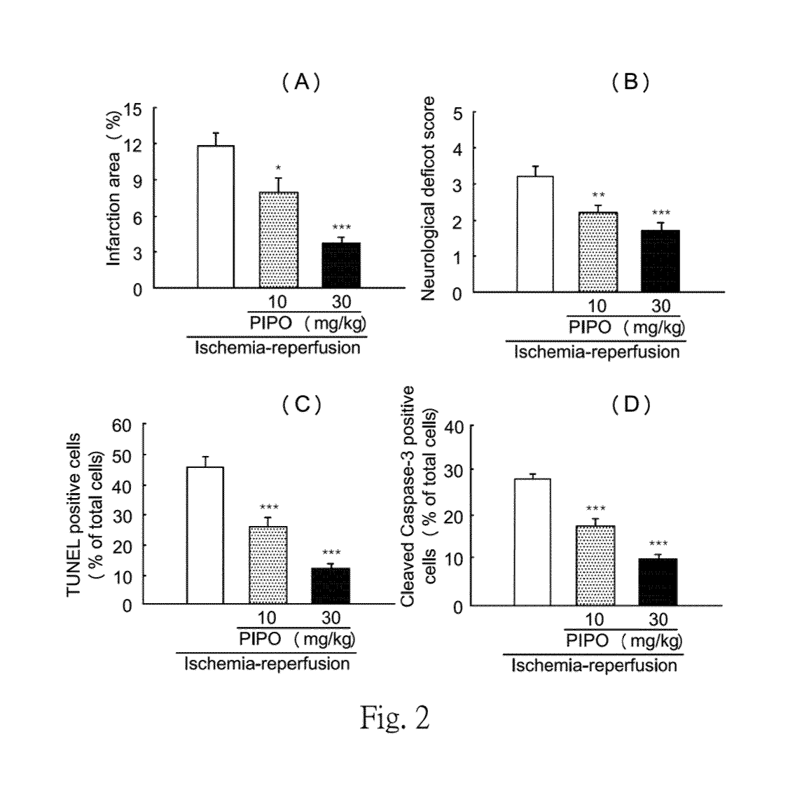

FIG. 1 and FIG. 2 illustrate the effect results of pipoxolan on cerebral infarction area in the brain of ischemic rat. Data are expressed as mean±SE. Differences among groups were analyzed using one-way analysis of variance (ANOVA) followed by Scheffe's test for individual comparisons. A p-value of <0.05 was considered statistically significant. Each vertical bars represented mean±S.E. *p<0.05, **p<0.01 and ***p<0.001 compared with control group. (n=6 in each group).

FIG. 1-(A) shows the effects of pipoxolan (10, 30 mg/kg p.o.) groups on cerebral infarct area at 24 hr after reperfusion. The pale area represents infarct tissue and the red area normal tissue. Scale bar=1 cm. Cerebral infarction in coronal sections was observed in the control group. Pipoxolan treatment (10 and 30 mg/kg) significantly reduced cerebral infarction as compared with the control group. Furthermore, FIG. 2-(A) represents the percent reduction of cerebral infarction by pipoxolan were 43.18% and 73.43% for 10 and 30 mg/kg:, respectively (***p<0.001).

FIG. 1-(B) is photomicrograph of TUNEL and cleaved caspase-3 positive staining in ischemic rats treated with/without pipoxolan. Scale bar=50 μm. Magnification 200×. Moreover, FIGS. 2-(C) and 2-(D) represent the percentage of positive cells in TUNEL and cleaved caspase-3 assay. TUNEL and cleaved caspase-3 positive cells were significantly decreased by pipoxolan treatment compared with control cells. Percent reduction of TUNEL positive cells by pipoxolan were 31.25% (10 mg/kg) and 46.88% (30 mg/kg), respectively. Pipoxolan induced reduction of cleaved caspase 3 positive cells were 37.18% (10 mg/kg) and 63.44% (30 mg/kg), respectively (***p<0.001). The result shows that pipoxolan treatment decreased positive cells of TUNEL and cleaved caspase-3 assay in a dose-dependent manner.

Furthermore, as shown in FIG. 2-(B), the neurological deficits in rats were 3.20±0.29, 2.20±0.20, 1.7±0.21, which was a reduction of 0, 31.25 and 46.88% by pipoxolan (0, 10 and 30 mg/kg p.o.), respectively.

II. Effects of Pipoxolan on Intimal Hyperplasia Caused by Vascular Injury

Animal Carotid Ligation Model

Male ICR mice, weighing 20 to 25 g, were used as a carotidligation model. Fifteen mice were randomly assigned to 3 treatment groups, five mice for each group. All mice were fasted overnight with free access to water, then anesthetized with zoletil® (25 mg/kg i.p.). Carotid ligation was performed by a midline neck incision and the left common carotid artery exposed. The carotid artery was completely ligated just proximal to the carotid bifurcation, and the right carotid artery served as a non-injured control artery. The incision was closed after ligation, and the animals were allowed to recover. On the day of carotid ligation, mice were randomized into 3 treatment groups and anesthetized as described. Group 1 mice were treated with saline and groups 2 and 3 treated with pipoxolan (10 mg/kg and 30 mg/kg, respectively). Subsequent pipoxolan and saline treatments were given by gastric gavage on the second day after carotid ligation and once daily for 28 days. All animals were euthanized on Day 29, and both right and left common carotid arteries were harvested, dehydrated in ethanol and xylene and embedded in paraffin for histomorphometric analysis.

(2) Hematoxylin-Eosin Staining and PCNA Antibody Staining of the Carotid Artery

Arterial sections (5 μm) of mice euthanized on Day 29 after ligation were stained by use of a hematoxylin-eosin solution. Images were digitized and analyzed with Image-Pro software. The areas of the lumen, internal elastic lamina (IEL), and external elastic lamina (EEL) were determined by computerized planimetry. The intima area was calculated by subtracting the luminal from the IEL area, and media area was determined by subtracting the IEL area from the EEL area. The ratio of intima to media area (I/M ratio) was calculated and analyzed. Arterial sections of animals from the different treatment groups were stained by using an immunofluorescence method to detect proliferating cell nuclear antigen (PCNA).

Results

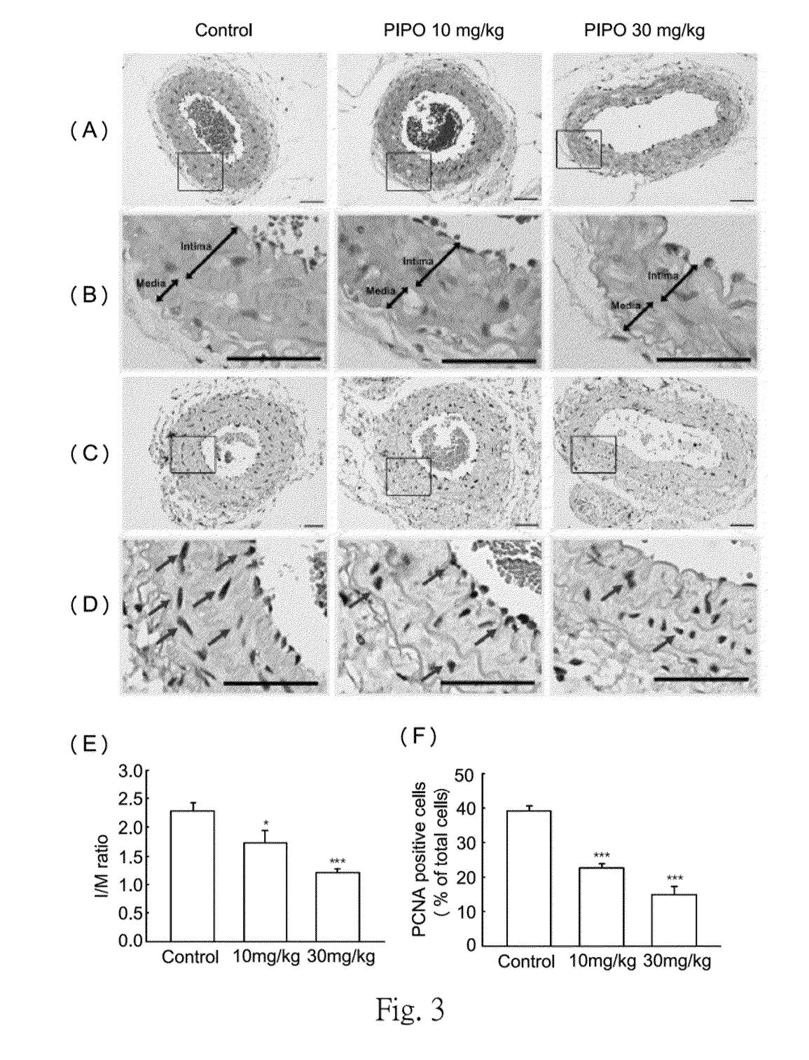

FIG. 3 shows the effects of pipoxolan on carotid-ligation induced intimal hyperplasia in mice. FIGS. 3-(A) and 3-(B) are photomicrographs of hematoxylin-eosin staining. FIGS. 3-(C) and 3-(D) represent PCNA-immunoreactivity staining of arterial section (200 X). FIG. 3E is ration of Intima/Media (I/M). FIG. 3-(F) is the percentage of PCNA positive cells per total cells. The arrow was indicating the PCNA positive cell. Scale bar=50 μm. Each vertical bars represented mean±S.E. *p<0.05 and ***p<0.001 compared with control group. (n=5 in each group).

After carotid-ligation in mice receiving pipoxolan or saline for 4 weeks, intimal hyperplasia morphology eras assessed by measuring the absolute lumen area, intima area (I), media area (M), and intima/media ratio (I/M). The morphology and I/M ratio were increased at 4 weeks following carotid-ligation. Mice treated with pipoxolan (10 mg/kg and 30 mg/kg p.o.) showed a significant reduction in carotid intimal hyperplasia compared with the control (saline) group. Percent reductions of I/M ratios were 24.21% (10 mg/kg, *p<0.05) and 47.20% (30 mg/kg, ***p<0.001) compared with the control group (FIGS. 3A, 3B and 3E). The number of proliferating cell nuclear antigen (PCNA)-positive cells was also inhibited by pipoxolan (10, 30 mg/kg) treatment. Percent inhibition was 42.11% (***p<0.001) and 62.40% (***p<0.001), respectively (FIGS. 3C, 3D and 3F).

III. Effects of Pipoxolan on VSMCs Migration

(1) Vascular Smooth Cell Line

The VSMCs cell line A7r5 was purchased from Bioresource Collection and Research Center (Hsinchu, Taiwan). A7r5 cells were plated onto 6-well plates in DMEM, supplemented with 10% FBS, 100 units/ml penicillin, 100 μg/ml streptomycin and 2 mM L-glutamine and grown at 37° C. under a humidified 5% CO2 and 95% air at one atmosphere.

(2) In Vitro Wound Healing Assay

The effects of pipoxolan on VSMCs migration were evaluated by a wound-healing assay. Platelet-derived growth factor (PDGF) form VMSCs can cause cell migration, which results in intimal hyperplasia. A7r5 cells were plated in 6-well plates (2×10⁵ cells per well) and damage was performed with a single scratch wound using a sterile micropipette tip. Cells were then incubated with or without PDGF-BB (30 ng/ml) and pipoxolan (5, 10, 15 μM) in serum-reduced DMEM medium (containing 0.5% fetal bovine serum). The extent of wound closure was determined using a phase contrast microscope at 24, 48, and 72 hr following wounding. Cell migration was expressed by the migration distance of drug-treated cells (mm) divided by the migration distance of untreated cells (mm).

(3) Transwell Migration Assay

Effects of pipoxolan on VSMCs migration were further investigated by a transwell migration chamber with a collagen-coated polycarbonate filter. A7r5 cells (2×10⁵ cells) were seeded on a transwell apparatus (a 6.5-mm polyethylene terephthalate membrane with 8-μm pores) and treated with pipoxolan (0, 5, 10 and 15 μM) in the presence of PDGF-BB (30 ng/ml) for 48 hr. The cells were then trypsinized, resuspended in 0.5% FBS medium. FBS/DMEM (10%) was added to the bottom chamber of each well as the chemoattractant. Cells were allowed to migrate for 8 hr through the membrane to the underside of the apparatus. Cells were then fixed with methanol for 10 min and stained with Giemsa solution for 30 min. The cells migrating to the lower outside of the insert membrane were counted manually using a microscope and the NIS-Elements software.

Results

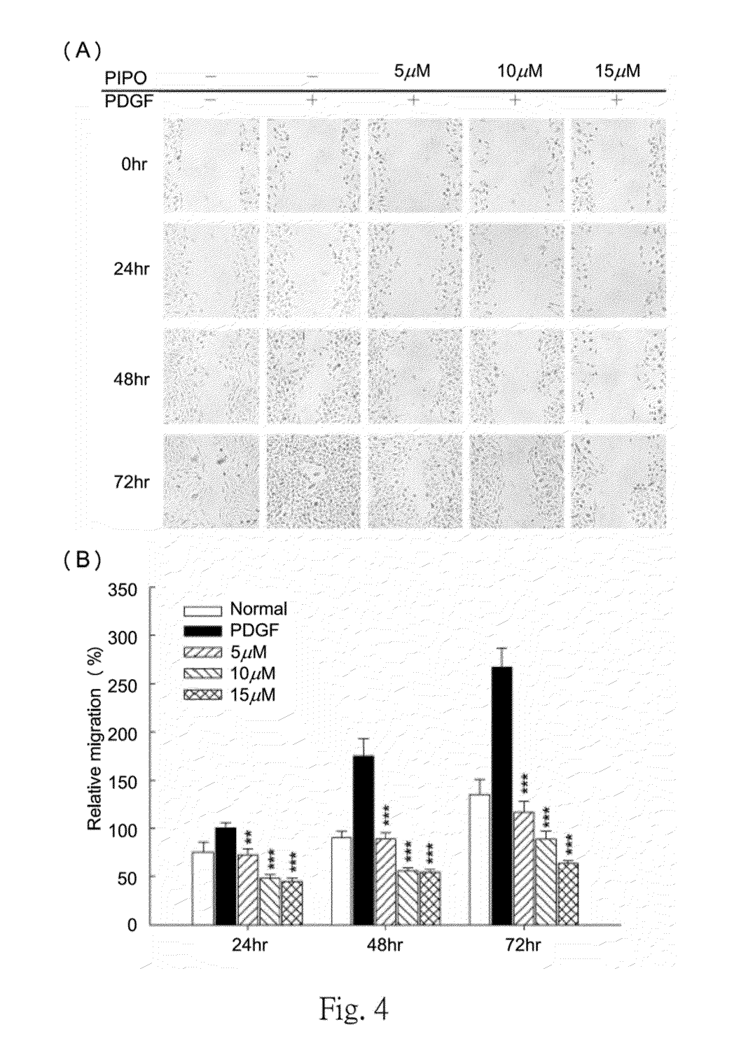

FIG. 4-(A) represents that a typical trace of pipoxolan (5, 10, and 15 μM) inhibited in response to PDGF-BB-induced VSMC migration. FIG. 4-(B) illustrates statistical differences in 24, 48 and 72 hr at different pipoxolan concentrations, respectively. Normal group was treated with vehicle. Each vertical bars represented mean±S.E. **p<0.01 and ***p<0.001 compared with PDGF-BB control group (n=6 in each group), Incubation of A7r5 cells with PDGF-BB-induced migration, which was significantly attenuated by pipoxolan at 24, 48 and 72 hr. Pipoxolan (5, 10 and 15 μM) markedly inhibited the PDGF-BB-induced cell migration in a dose-dependent manner. The inhibitory effects on A7r5 cell migration were further confirmed by a transwell migration assay.

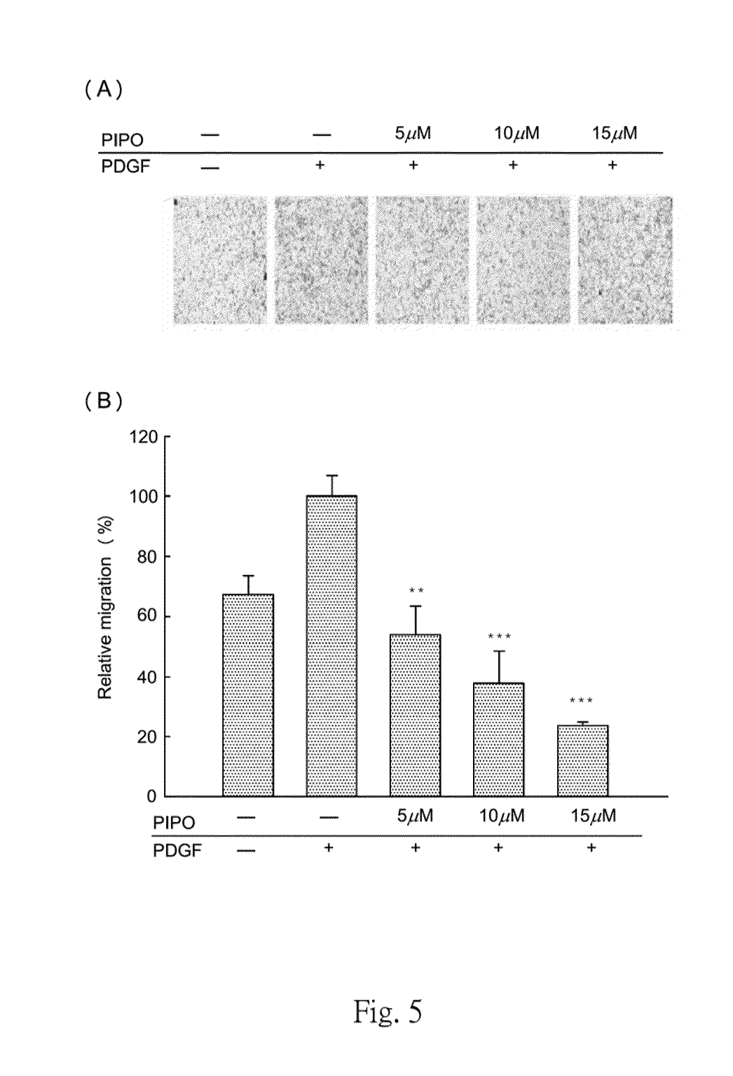

FIG. 5 shows the effects of pipoxolan on PDGF-BB-induced VSMC migration by transwell assay. The ability of A7r5 cells to move across the membrane was significantly reduced by pipoxolan. Percent inhibition of cells treated with pipoxolan (5, 10 and 15 μM) was 46.2% (**p<0.01), 62.2% (***p<0.001) and 76.5% (***p<0.001) compared with the positive control (PDGF-BB) cells, respectively.

Because VSMCs migration and proliferation are associated with intimal thickening, the data of FIG. 1 to FIG. 5 indicate that pipoxolan can counter effects of proliferation, which caused by ischemic-reperfusion-induced cerebral infarction and carotid artery ligation by inhibiting VSMCs migration.

IV. Effects of Pipoxolan on Protein Expression Level in Signal Protein of VSMCs Migration

The role of matrix metalloproteinase-2/9 (MMP-2/9) and the Ras/MEK/ERK signaling pathways were used to evaluate the effect of pipoxolan on VSMCs migration. The protein expression levels were determined by Western blot analysis. Each vertical bars represented mean±S.E. **p<0.01 and ***p<0.001 compared with control group (n=6 in each group).

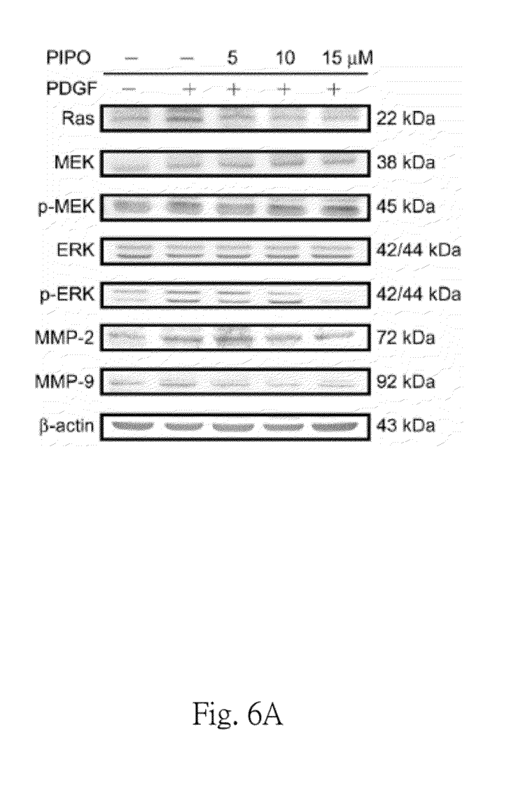

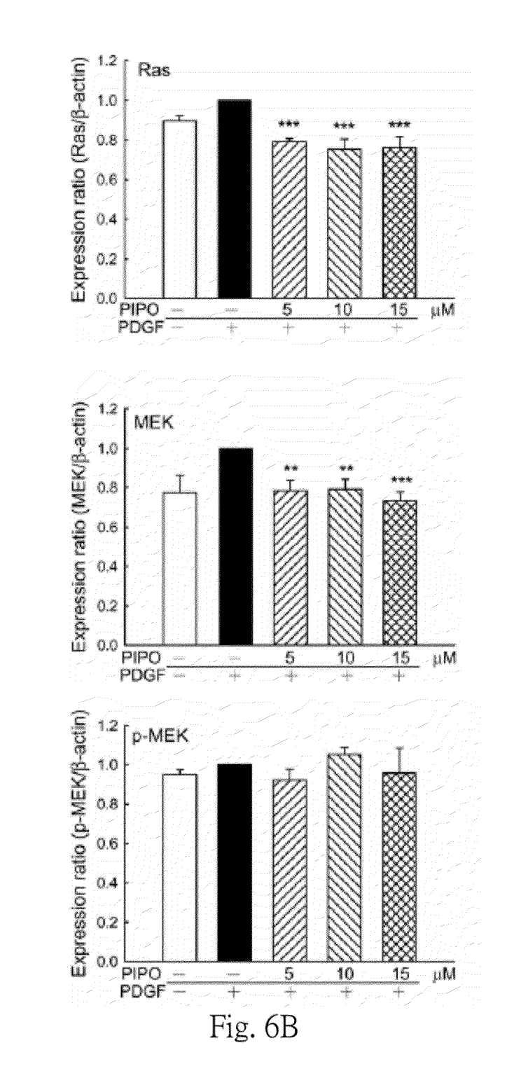

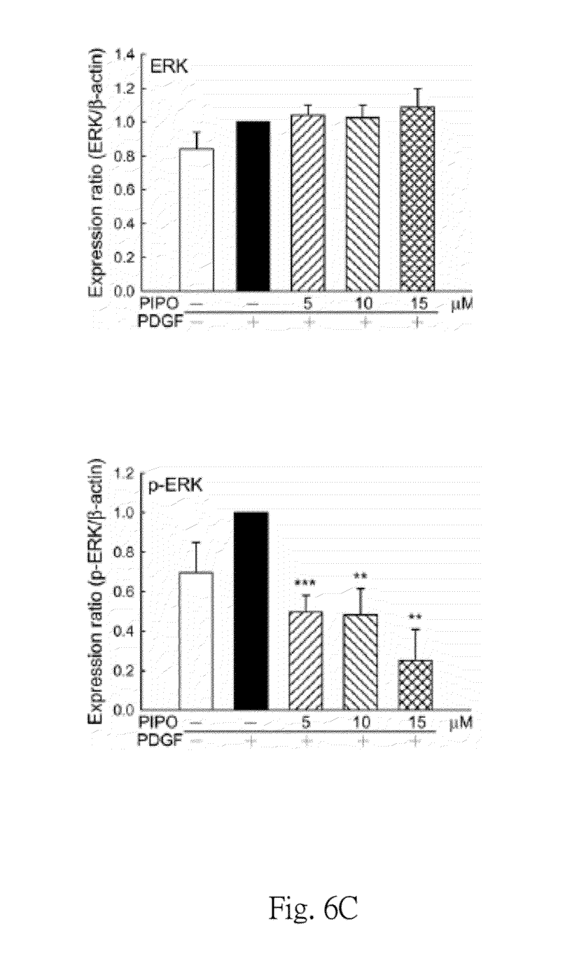

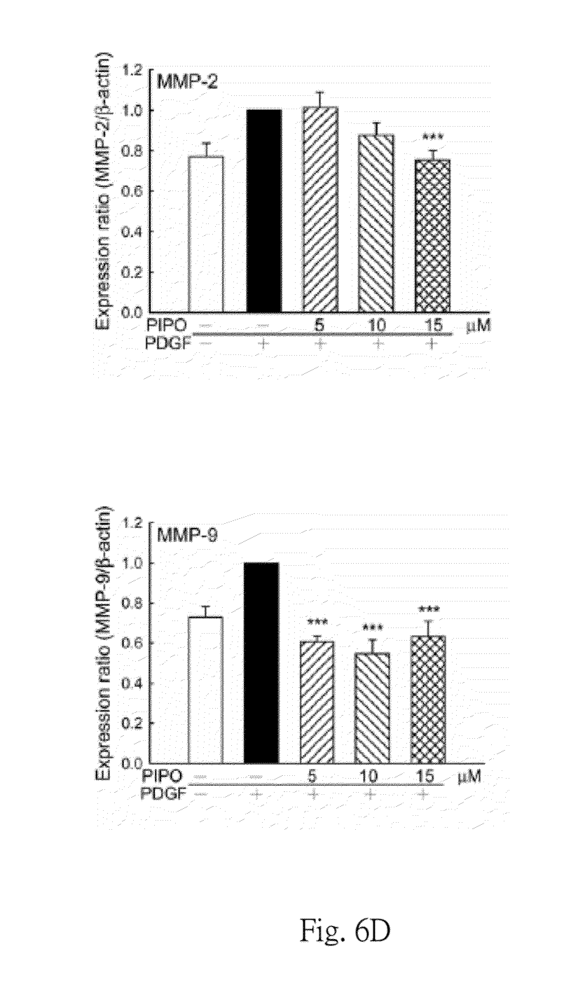

FIG. 6A to FIG. 6D represent the effect results of pipoxolan on levels of the candidate signaling proteins MMP-2/9, Ras, MEK, p-MEK, ERK and p-ERK in PDGF-BB-stimulated VSMCs. Pipoxolan at a concentration of 5, 10, 15 μM significantly reduced protein levels of Ras (21%, 24.75% and 24%, respectively ***p<0.001); MEK (21.67%, 21% and 26.67%, respectively ***p<0.001); and p-ERK (50.33%, 51.67% and 75%, respectively ***p<0.001). MMP-2 protein levels were reduced by pipoxolan (10, 15 μM) 12.4% and 24.4%, respectively ***p<0.001. MMP-9 levels were significantly inhibited by pipoxolan (5, 10, 15 μM) at 39.4%, 45.4% and 36.6%, respectively (***p<0.001). As shown in FIG. 6A to FIG. 6D, pipoxolan significantly does-dependent reduced protein levels of Ras, MEK, p-ERK, expression of MMP-2 and MMP-9.

A possible mechanism of VSMCs migration is via secretion of matrix metalloproteinases (MMPs). MMPs are increased at the site of vascular injury, and inhibitors of MMPs reduce VSMCs migration after vascular injury both in vitro and in vivo. Therefore a medicament that reduces VSMCs proliferation and migration could be beneficial in treating vascular disease. According to the results of FIG. 1 to FIG. 6D, pipoxolan may inhibit VSMCs migration by inhibiting the proteins of signaling pathways in VSMCs migration. Thus pipoxolan can effectively reduce intimal thickening caused by VSMCs migration and proliferation. Moreover, the results also indicate that treatment with pipoxolan has protective effect on cerebral ischemia and carotid artery ligation-induced intimal hyperplasia. Treatment with pipoxolan also has inhibitory effect on the ischemia/reperfusion induced VSMCs migration caused by vascular injury. Thus the multi-faced effect of pipoxolan could be potentially efficacious in preventing cerebrovascular disease in high risk individuals, and it could be useful in treating patients with vascular disease.

According to the above test results, pipoxolan can be used as a pharmaceuticals or pharmaceutical compositions for treating vascular diseases to alleviate cerebral vascular pathological condition of a subject. The pharmaceuticals or pharmaceutical compositions, according to a conventional pharmaceutically process prepared, may contain pharmaceutically acceptable adjuvants which is compatible with the other ingredients of the formulation and is compatible with the living body. The term “pharmaceutically acceptable adjuvant” or “pharmaceutically acceptable carrier means a pharmaceutically acceptable material, composition or vehicle, such as liquid or solid filler, diluent, excipients, a solvent or encapsulating material. It can be used in carrying or transporting the subject composition from the organ or part of the body to another organ or part of the body. The term acceptable” means a carrier which may contain other compatible components of composition. The carrier may be solid, semi-solid, liquid, cream or capsule form.

According to one embodiment of the present disclosure, a pharmaceutical composition including the pipoxolan as the main active ingredient is used to reduce cerebral vascular pathology can be administered via oral (e.g., oral capsules, suspensions or tablets drug) or parenteral methods. Parenteral administrations include intramuscular, intravenous, subcutaneous or intraperitoneal injections administered in a systematic way. Alternatively, it may be administered via penetrate through the involucra or intrarectal administration. The administration may be administered pharmaceutical alone or in combination with other conventional pharmaceutically acceptable adjuvants. In the preferred embodiment, the pharmaceutical composition of the present disclosure is administered to a subject via the oral route (e.g., through food).

in terms of orally administered, the pharmaceutical composition including the pipoxolan as the main active ingredient are the medicine tablets which contain various adjuvants (such as microcrystalline cellulose, calcium carbonate, dicalcium phosphate and glycine), various disintegration agents (such as starch, alginic acid and certain silicates), particles adhesives (e.g., polyvinylpyrrolidone, sucrose, gelatin and acacia gum). They also contain lubricants such as magnesium stearate sodium lauryl sulfate and talc. This solid formula can be used as the filling material for gelatin capsules, or preferred materials which include lactose or milk sugar and high molecular weight polyethylene glycols. When the administration is oral suspensions and/or elixirs, the active ingredient may be combined with various sweetening or flavoring agents, coloring matter or dyes in the formulation. Besides, the emulsifier and/or suspended and diluents such as water, alcohol, propylene glycol, glycerin may be added to the oral suspensions and/or elixirs.

In terms of parenteral administered, the pharmaceutical compositions of the present disclosure are liquid formulations, which may be sterile solutions or suspensions for intravenous injection, intramuscular injection, intraperitoneal injection, or subcutaneous administration. The liquid diluents which are used for manufacture above sterile solution or suspension include, but are not limited to, 1,3-butylene glycol, mannitol, water, Ringer's solution, isotonic sodium chloride solution, fatty acid (e.g. oleic acid) and the ester derivative thereof, or a pharmaceutically acceptable natural oils (such as olive or canola oil). The oily solutions or suspensions may also contain dispersing agents for diluting alcohol or carboxymethyl. Moreover, the oily solutions or suspensions may contain surfactants (eg, Tweens or Spans series), emulsifying agents, or agents which are often used to enhance bioavailability.

The above pharmaceuticals or pharmaceutical compositions of the present disclosure are also made to a variety of mucosal application dosage formulations, such as buccal and/or the sublingual pharmaceutical dosage unit to deliver the drug to penetrate the oral mucosa. The mucosal application dosage formulations can use various polymer adjuvants, which are biodegradable and pharmaceutically acceptable. The polymer adjuvants have effect of drug absorption and desired release pattern of pharmaceutical compositions, and they are compatible with the active ingredient or other ingredients of buccal and/or sublingual pharmaceutical dosage unit. Generally, the polymer adjuvants as described comprise a hydrophilic polymer, which may adhere to the wet surface of the oral mucosa. The embodiments of polymer adjuvants include, but are not limited to, acrylic acid polymers and copolymers, hydrolyzed polyvinyl alcohol, polyethylene oxide, polyacrylate, vinyl polymers and copolymers, polyvinyl pyrrolidine, dextran, guar gum, pectin, starch, and cellulosic polymers.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for treating a pathological condition caused by proliferation and migration of vascular smooth muscle cells (VSMCs), comprising administering an effective amount of pipoxolan.

2. The method according to claim 1, wherein the pathological condition caused by proliferation and migration of VSMCs is a vascular injury.

3. The method according to claim 2, wherein the vascular injury is a cerebrovascular accident.

4. The method according to claim 3, wherein the cerebrovascular accident is an infarction caused by ischemia.

5. The method according to claim 1, wherein the pathological condition caused by proliferation and migration of VSMCs is an intimal hyperplasia.

6. The method according to claim 5, wherein the intimal hyperplasia is an atherosclerotic stenosis.

7. The method according to claim 5, wherein the intimal hyperplasia is caused by vascular injury.

8. The method according to claim 1, wherein the pathological condition caused by proliferation and migration of VSMCs is a cerebral ischemia.

9. The method according to claim 1, wherein the pathological condition caused by proliferation and migration of VSMCs is a stroke.

10. A pharmaceutical composition for reducing a cerebrovascular pathological condition in a subject, comprising a therapeutically effective amount of a pipoxolan HCl and a pharmaceutically acceptable carrier.

11. The pharmaceutical composition according to claim 10, wherein the cerebrovascular pathological condition is a cerebrovascular infarction caused by ischemia.

12. The pharmaceutical composition according to claim 10, wherein the cerebrovascular pathological condition is a cerebrovascular infarction caused by atherosclerosis.

13. The pharmaceutical composition according to claim 10, wherein the pharmaceutically acceptable carrier comprises at least one additional component selected from the group consisting of: an excipient, a solvent, an emulsifier, a suspending agent, a decomposer, a cohesive material, a stabilizer, a conservative, an absorption delaying agent and a liposome.
